(19) United States
(12) Patent Application Publication
(10) Pub. No.: US 2019/0209465 A1
(43) Pub. Date: Jul. 11, 2019

(54) PROCESS FOR PREPARING PHARMACEUTICAL OPHTHALMIC COMPOSITIONS OF BRINZOLAMIDE

(71) Applicant: PHARMATHEN S.A., PALLINI-ATTIKIS (GR)

(72) Inventors: EVANGELOS KARAVAS, PALLINI ATTIKIS (GR); EFTHYMIOS KOUTRIS, PALLINI ATTIKIS (GR); VASILIKI SAMARA, PALLINI ATTIKIS (GR); IOANNA KOUTRI, PALLINI ATTIKIS (GR); ANASTASIA KALASKANI, PALLINI ATTIKIS (GR); AMALIA DIAKIDOU, PALLINI ATTIKIS (GR); ANDREAS KAKOURIS, PALLINI ATTIKIS (GR); RUMIT SHAH, PALLINI ATTIKIS (GR)

(73) Assignee: PHARMATHEN S.A., PALLINI-ATTIKIS (GR)

(21) Appl. No.: 16/308,567

(22) PCT Filed: Jul. 1, 2016
(86) PCT No.: PCT/EP2016/025071

§ 371 Date: Dec. 10, 2018

(51) Int. Cl. A61K 9/00; A61K 31/542; A61K 9/107; A61K 47/14; A61K 47/34; A61K 47/10; A61K 47/38; A61K 47/24; A61K 47/18; A61K 47/02

(52) U.S. Cl. CPC A61K 9/0048; A61K 31/542; A61K 9/1075; A61K 47/14; A61K 47/02; A61K 47/10; A61K 47/38; A61K 47/24; A61K 47/18; A61K 47/34

(57) ABSTRACT

The present invention relates to the field of drug delivery and, particularly, to alternative processes for preparing ophthalmic compositions of Brinzolamide or pharmaceutical acceptable salts thereof.

### TECHNICAL FIELD OF INVENTION

The present invention relates to a process for preparing a stable ophthalmic pharmaceutical formulation of a carbonic anhydrase inhibitor. More particularly, it relates to alternative processes for preparing a stable ophthalmic pharmaceutical formulation of Brinzolamide.

### BACKGROUND OF THE INVENTION

Glaucoma is a disease, usually caused by high intraocular pressure, which leads to disruption of normal eye function and subsequently, degeneration of the eye. The damage can be extended to the optic nerve head and result in irreversible loss of the eyesight and if left untreated it could lead to irreversible blindness. Nowadays, it is believed by the majority of ophthalmologists that the increased intraocular pressure (also known as ocular hypertension) is the earliest phase in the onset of glaucoma. Later symptoms include optic nerve head damage and the characteristic glaucomatous visual effects.

The early methods for the treatment of glaucoma included the drug pilocarpine, which produced undesired local side effects. More recently new regimes have been employed for the treatment of ocular hypertension and glaucoma.

It is known that carbonic anhydrase inhibitors are used for the treatment of ocular hypertension related to glaucoma. The drugs that belong to this family inhibit the enzyme carbonic anhydrase and thus, reduce the contribution of the aqueous humor formation made by the carbonic anhydrase pathway. However, these drugs cannot be used via a systemic route because then they inhibit the enzymatic activity of carbonic anhydrase throughout the entire body. In general, the enzyme carbonic anhydrase plays a major role in regulating pH and fluid levels in the human body by converting carbon dioxide to carbonic acid and bicarbonate ions.

Targeting of the carbonic anhydrase inhibitor to the desired ocular tissue diminishes or even eliminates the side effects caused by the inhibition of carbonic anhydrase in the entire body, which can be as severe as metabolic acidosis or less severe, like numbness, vomiting, tingling, general malaise and the like.

Brinzolamide, a carbonic anhydrase inhibitor, is the chemical molecule designated as (R)-4-ethylamino-3, 4-dihydro-2-(3-methoxy) propyl-2H-thieno[3, 2-e]-1, 2-thiazine-6-sulfonamide 1, 1 dioxide. It has been found to reduce intraocular pressure with fewer side effects compared to the earlier glaucoma treatments. Brinzolamide is a white to almost white powder with a melting point at 131° C. Furthermore, it is insoluble in water and slightly soluble in alcohol and methanol.

U.S. Pat. No. 4,797,413 & U.S. Pat. No. 4,847,289 & U.S. Pat. No. 4,731,368 disclose topically administered thiophene sulfonamides which lower IOP by inhibiting carbonic anhydrase.

EP-B-941094 discloses a process for the preparation of Brinzolamide suspension and the use of Tyloxapol® and Triton® X-100 as a surfactant.

EP-A-2394637 discloses a process for the manufacture of sterile ophthalmic suspensions comprising Brinzolamide, characterized in that it comprises a step of sterilization of Brinzolamide by gamma irradiation or ethylene oxide.

Although each of the above patents represents an attempt to overcome the low aqueous solubility problems associated with topical pharmaceutical compositions comprising Brinzolamide, a need still exists for the development of new and effective drug delivery systems for water insoluble or sparingly soluble drugs like Brinzolamide having desired bioavailability and degree of comfort to the patient.

### SUMMARY OF THE INVENTION

The main object of the present invention is to develop new and effective drug delivery systems for water insoluble or sparingly soluble drugs, in particular Brinzolamide or pharmaceutical acceptable salts thereof that overcome the deficiencies of the prior art.

Therefore, the objective of the present invention is to provide an ophthalmic preparation comprising Brinzolamide that matches the characteristics of the marketed product but has better patient tolerability and bioavailability.

A major aspect of the present invention is to formulate thermodynamically stable oil-in-water microemulsion and micelle solubilization.

Another aspect of the present invention is to provide simple and cost effective processes for preparing ophthalmic preparations comprising Brinzolamide or pharmaceutical acceptable salts thereof.

According to another embodiment of the present invention Decrease pH and High Pressure Homogenization methods are used for the preparation of microemulsion.

According to further embodiment of the present invention Decrease pH and High Pressure Homogenization methods are used for the preparation of micellar solubilization.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient is considered to be “stable” if said ingredient degrades less or more slowly than it does on its own and/or in known pharmaceutical compositions.

Ocular administration of drugs is primarily associated with the need to treat ophthalmic diseases. Eye is the most easily accessible site for topical administration of a medication. Ophthalmic preparations are sterile products essentially free from foreign particles, suitably compounded and packaged for instillation into the eye. They are easily administered by the nurse or the patient himself, they have quick absorption and effect, less visual and systemic side effects, increased shelf life and better patient compliance.

As already stated the object of the present invention is to develop ophthalmic preparations comprising Brinzolamide or pharmaceutical acceptable salts thereof of similar characteristics with marketed product but in alternative form in order to achieve better patient tolerability and desired bioavailability.

The marketed product is in the form of micro-suspension. Such form not only is difficult to manufacture but also has other disadvantages such as slower rate of absorption when compared to solutions; it may develop residue in the eyelashes after dose administration; feeling of foreign body in eye, blurred vision and swift humour secretion resulting quick wash out of drug even before it acts in case of suboptimal dose administration; problems associated with physical stability, sedimentation and compaction.

Recently, various nanonization strategies have emerged to increase the bioavailability of numerous drugs that are poorly soluble in water and during the past decade several drug formulations have been clinically approved or are under clinical investigation. Major research efforts have been focused on the development of enabling nanoformulation technologies to improve product properties while keeping production cost as low as possible. Nanoformulations may exist in various forms such as nanocrystals, microemulsions and micelles.

A dispersion of oil in water (o/w) can be defined as either a macroemulsion or a microemulsion. A macroemulsion is cloudy turbid composition with an oil-droplet size of 0.5 to 100 μm. Macroemulsions are usually unstable. In contrast to macroemulsion systems, microemulsions are isotropic systems consisting of oil and water. Appropriate emulsifiers may form spontaneously and are therefore thermodynamically stable. For this reason, microemulsion systems theoretically have an infinite shelf life under normal conditions in contrast to the limited life of macroemulsions.

A microemulsion is a translucent to transparent composition having droplet size ranges from 10-200 nm, and has very low oil/water interfacial tension. However, one must choose materials that are biocompatible, non-toxic, clinically acceptable, and use emulsifiers in an appropriate concentration range to form stable microemulsions.

Examples of emulsions are included in U.S. Pat. Nos. 4,914,088; 5,294,607; 5,371,108 and 5,578,586.

Nano-suspension, in comparison to micronized suspension, has significantly greater surface area. The increase in surface area enhances the bioavailability and degree of comfort when administered in eye.

The terms ‘surfactant’ and ‘emulsifier’ as used in the present invention are identical and mean an amphiphilic compound with the following properties: it has hydrophobic groups and hydrophilic groups; it can form micelles; it is capable of migrating to the water surface where the insoluble hydrophobic alkyl chains may extend out of the bulk water phase, either into the air or, if water is mixed with oil, into the oil phase, while the water soluble head group remains in the aqueous phase; it can stabilize colloidal dispersion of nano sized drug particles in the aqueous phase; it can solubilize water insoluble substances through micellar solubilization.

The term ‘microemulsion’ as used herein means a thermodynamically stable dispersion of two immiscible liquids, stabilized by surfactants; it is typically clear because the dispersed droplets are less than 200 nanometers in diameter. The components to generate microemulsion include, but are not limited to oil, water, surfactant and co-surfactant.

The term ‘nanosuspension’ as used herein means a stable dispersion of nanosized drug particles, stabilized by surfactants; where the average diameter of dispersed drug particles are less than 1 micrometer, in particular between 100-800 nm, more particular between 100-500 nm, in particular between and about 100-350 nm, more in particular about 300 nm.

The term ‘percent transmission’ as used herein is defined as follows: when light is allowed to pass through a solution, the percentage of incident light which is transmitted through the solution is referred to as “percent transmission”. The “percent transmission” generally defines the visible clarity of the composition.

The term “homogenization” for microemulsion refers to processing an oil phase into a number of ultrafine droplets and dispersing and maintaining them in an aqueous phase.

The term “homogenization” for nanosuspension refers to processing macro or micron size drug particles into stable nanosized particles and dispersing and maintaining them in an aqueous phase.

The present invention provides a nanosuspension of a poorly soluble drug with improved bioavailability made using high-shear bead milling or high pressure homogenizer or microfluidizer processor, wherein said nanosuspension is suitable for long term storage. A method of preparation of an ophthalmic nanosuspension of a poorly soluble drug with improved bioavailability consists of breaking down process and building up process alone or in combination. In certain embodiments, the method also comprises a step of stirring the drug, which has been micronized, in an aqueous surfactant excipient solution for wetting and dispersing, followed by a step of passing the resulting mixture through a high-shear bead milling or high pressure homogenizer or microfluidizer processor.

The present invention also provides oil in water micro-emulsion, which comprises pharmaceutically acceptable oil as internal phase uniformly distributed in buffered water as external phase with the help of surfactant.

Processes according to the present invention aim at manufacturing thermodynamically stable oil-in-water microemulsion or nanosuspension or micelle solubilization, preferably having a mean size generally of more than about 10 nm and less than about 500 nm. For example, the mean size of the droplets or dispersed particles may be of more than about 10 nm and less than about 300 nm, preferably less than about 200 nm.

For homogenization, known means such as a homomixer, bead milling, a homogenizer, a high-pressure homogenizer, an ultra-high-pressure homogenizer (microfluidizer) and the like can be used alone or in combination. Other additives such as tonicity agent, buffering agent, preservative and the like may also be dissolved in an aqueous phase or added after homogenization. Solvents or cosolvents that may be selected from a group of alcohols, such as ethanol, glycols such as ethylene glycol, propylene glycol, polyethylene glycol, glycofurol and the like may also be used.

In another aspect the present invention relates to processes for manufacturing pre-concentrates of ophthalmic oil-in-water emulsions, nanosuspension and micellar solubilization. The process for manufacturing pre-concentrates of ophthalmic oil-in-water emulsions, preferably of ophthalmic oil-in-water microemulsions or submicroemulsions, comprise a step of homogenizing an oil phase with an aqueous phase and at least one surfactant to obtain a pre-concentrate of an oil-in-water emulsion. A pre-concentrate prepared by such a process generally has content in oil that is higher than the content in oil of the final oil-in-water emulsion prepared by dilution of the pre-concentrate.

The process for manufacturing pre-concentrates of ophthalmic nanosuspension comprises a step of homogenizing macro or micron size drug particles with an aqueous phase and at least one surfactant to obtain a pre-concentrate of stable nanosized particles and dispersing and maintaining them in an aqueous phase. A pre-concentrate prepared by such a process generally has higher concentration of nanosized drug particles than the final formulation prepared by dilution of the pre-concentrate.

More specifically, in processes of the present invention, a pre-concentrate of a desired oil-in-water emulsion is produced by homogenizing an oil phase comprising oil that is suitable for ophthalmic use, with an aqueous phase and at least one surfactant. Oils suitable for ophthalmic use include, but are not limited to, castor oil, isopropyl myristate, MCT, mineral oils, vegetal oils, and any combinations of these oils that are well tolerated at the eye level. As used herein, the term “MCT” refers to medium chain triglycerides. Medium chain triglycerides generally have a high solubility in water, are not significantly susceptible to oxidation, and are well suited for ophthalmic applications. Examples of vegetal oils include, but are not limited to, cotton seed, ground nut, corn, germ, olive, palm, soybean, and sesame oils. Examples of mineral oils include, but are not limited to, silicone and paraffin.

Surfactants suitable for use in processes of the present invention may be non-ionic surfactants, cationic or anionic surfactants. Examples of non-ionic surfactants that can be used in processes of the present invention include, but are not limited to, polyoxyethylene castor oil derivatives, derivatives of cremophors (e.g. kolliphor EL, and cremophor RH), polysorbates (e.g. tween 80), tyloxapol poloxamers sorbitan esters, polyoxyl stearates, and combinations thereof. Examples of cationic surfactants that are suitable for use in the present invention include, but are not limited to, C10-C24 primary alkylamines, tertiary aliphatic amines, quaternary ammonium compounds selected from the group comprising lauralkonium halide, cetrimide, hexadecyl-trimethylammonium halide, tetradecyltrimethyl-ammonium halide, dodecyltrimethyl-ammonium halide, cetrimonium halide, benzethonium halide, behenalkonium halide, cetalkonium halide, cetethyldimonium halide, cetylpyridinium halide, benzododecinium halide, chlorallyl methenamine halide, myristalkonium halide, stearalkonium halide or a mixture of two or more thereof, halide being preferably chloride or bromide, cationic lipids, amino alcohols, biguanide salts selected from the group comprising or consisting of chlorhexidine and salts thereof, polyaminopropyl biguanide, phenformin, alkylbiguanide or a mixture of two or more thereof, cationic compounds selected from 1,2-dioleyl-3-trimethyl-ammoniumpropane, 1,2-dioleoyl-sn-glycerophosphatidyl-ethanolamine, cationic glycosphingo-lipids or cationic cholesterol derivatives and any combinations thereof. Examples of anionic surfactants that are suitable for use in the present invention include, but are not limited to, lecithin, bile salts, fatty acids, and any combinations thereof.

Cosurfactants are usually added to a process to enhance the effectiveness of a surfactant; polyethylene glycols (e.g. PEG 200) and 2-(2-ethoxyethoxy)-ethanol (Transcutol) may be used as cosurfactants in the present invention.

The ophthalmic composition of the present invention may further comprise pharmaceutically acceptable excipients conventional to the pharmaceutical art such as osmotic/tonicity adjusting agents, one or more pharmaceutically acceptable buffering agents and pH-adjusting agents, viscosity enhancing agents, penetration enhancing vehicles and other agents.

The compositions of the present invention may contain a water-soluble polymer to increase the stability of emulsion particles. Examples of the water-soluble polymer include hydroxy ethyl cellulose, hydroxy propyl cellulose, povidone (polyvinylpyrrolidone), polyvinyl alcohol, methyl cellulose, hydroxy propyl methyl cellulose, carboxy methyl cellulose, and salts thereof and the like. The water-soluble polymer may be comprised in a range from 0.001% w/v to 3% w/v.

The ophthalmic compositions of the present invention are isotonic with respect to the ophthalmic fluids present in the human eye. These compositions are characterized by osmolality of 250-375 mOsm/kg. Osmolality is adjusted by addition of an osmotic/tonicity adjusting agent. Osmotic agents may be selected from sodium chloride, potassium chloride, calcium chloride, sodium bromide, sodium phosphate. sodium sulfate, mannitol, glycerol, sorbitol, propylene glycol, dextrose, sucrose, polyethylene glycols (PEG), PEG-400, PEG-200, PEG300 and the like, and mixtures thereof.

The compositions of the present invention may additionally contain various additives such as stabilizer, chelating agent, pH adjusting agent, thickener and the like. Examples of the chelating agent include disodium edetate, citric acid and salts thereof. In order to achieve, and subsequently maintain, an optimum pH, the ophthalmic composition may contain a pH adjusting agent and/or a buffering agent. The preferred range of pH for an ophthalmic formulation is about 3.00 to about 8.00, and the most preferred pH is about 4.00-8.00. The ophthalmic compositions of the present invention comprise pharmaceutically acceptable pH adjusting agents that may be selected from the group comprising acetic acid or salts thereof, boric acid or salts thereof, phosphoric acid or salts thereof, citric acid or salts thereof, tartaric acid or salts thereof, sodium hydroxide, potassium hydroxide, sodium carbonate, sodium hydrogen carbonate, trometamol, arginine, lysine, histidine, guanine and the like and mixtures thereof. Particularly, preferred pH adjusting agents that may be used in the present invention include acetic acid, hydrochloric acid, tromethamine, arginine and sodium hydroxide. These agents are used in amounts necessary to produce a pH ranging from about 4.50 to about 8.00.

The composition of the present invention may also contain a preservative as long as it does not markedly decrease the storage stability of Brinzolamide. Preservatives selected from the group comprising benzalkonium chloride (BAK), benzethonium chloride, benzyl alcohol, edetate disodium, borates, pyruvates, parabens, stabilized oxychloro compounds, sorbic acid/potassium sorbate, polyaminopropyl biguanide, polyquarternium-1, polyhexamethylene biguanide (PHMB), PVP-Iodine complex, metal ions, peroxides, aminoacids, arginine, tromethamine, cetrimide, chlorhexidine, chlorobutanol, mercurial preservatives, or phenylmercuric nitrate, phenylmercuric acetate, thimerosal, phenylethyl alcohol, and mixtures thereof may be included within the scope of the present invention. These compounds are generally regarded as safe and are recommended for long term use.

Microemulsions are prepared by the spontaneous emulsification method (phase titration method) and can be depicted with the help of phase diagrams. Construction of phase diagram is a useful approach to study the complex series of interactions that can occur when different components are mixed.

Microemulsions are formed along with various association structures (including emulsion, micelles, lamellar, hexagonal, cubic, and various gels and oily dispersion) depending on the chemical composition and concentration of each component. The understanding of their phase equilibriums and demarcation of the phase boundaries are essential aspects of the study. As quaternary phase diagram (four component system) is time consuming and difficult to interpret, pseudo ternary phase diagram is often constructed to find the different zones including microemulsion zone, in which each corner of the diagram represents 100% of the particular component. The region can be separated into w/o or o/w microemulsion by simply considering the composition that is whether it is oil rich or water rich. Observations should be made carefully so that the metastable systems are not included.

Phase inversion of microemulsions occurs upon addition of excess of the dispersed phase or in response to temperature. During phase inversion drastic physical changes occur including changes in particle size that can affect drug release both in vivo and in vitro. These methods make use of changing the spontaneous curvature of the surfactant. For non-ionic surfactants, this can be achieved by changing the temperature of the system, forcing a transition from an olw microemulsion at low temperatures to a w/o microemulsion at higher temperatures (transitional phase inversion). During cooling, the system crosses a point of zero spontaneous curvature and minimal surface tension, promoting the formation of finely dispersed oil droplets. This method is referred to as phase inversion temperature (PIT) method. Instead of the temperature, other parameters such as salt concentration or pH value may be considered as well instead of the temperature alone.

Additionally, a transition in the spontaneous radius of curvature can be obtained by changing the water volume fraction. By successively adding water into oil, initially water droplets are formed in a continuous oil phase. Increasing the water volume fraction changes the spontaneous curvature of the surfactant from initially stabilizing a w/o microemulsion to an o/w microemulsion at the inversion locus. Short-chain surfactants form flexible monolayers at the o/w interface resulting in a bicontinuous microemulsion at the inversion point.

Nanosuspension was prepared by making slurry of Brinzolamide in surfactant solution followed by particle size reduction based on high energy input by HPH, bead milling, microfluidics or any other means.

High pressure homogenization is considered as a safe technique for producing nanosuspensions. The combined forces of cavitation, high shear, and collisions lead to fracture of the drug microparticles into nanosized particles. Homogenization pressure, number of homogenization cycles, hardness of drugs, and temperature (when thermosensitive drugs are processed) are factors that influence the physical characteristics (such as particle size) of the resulting nanosuspension.

High shear/speed mixing of a coarse emulsion or macro-suspension can be performed using rotor-stator, high pressure or ultrasonic devices. In the present invention HP homogenization is preferred. In HP homogenization, fluid is forced at high pressure by means of a plunger pump through a very narrow channel. Depending on the type of homogenizer, the fluid may then collide head on with another high velocity stream or hit a hard-impact ring. Droplet size is reduced by cavitation, high shear forces, and high speed collisions with other droplets. Pressure, temperature and number of passes are parameters that can be controlled and influence the efficiency and magnitude of size reduction. HP-homogenizers of piston gap type consist of one or two piston intensifiers able to generate high pressure, and HP-valve equipped with ceramic needles and seat of specially engineered design. In such HP-homogenizers the fluid under pressure is forced through a small orifice of some micrometers width, the HP-valve gap. The fluid accelerates over a very short distance to very high velocity and the resulting strong pressure gradient between the inlet and outlet of the HP-valve generates intense shear forces and extensional stress through the valve gap. Cavitation, turbulence and impact with solid surfaces take place at the outlet of the valve gap. Due to shear effects and conversion of kinetic energy into heat, the fluid travelling through the HP-valve is accompanied by short-life heating phenomena that can be controlled by efficient cooling devices. All the mechanical forces are expected to disrupt particles down to the submicron range. This type of equipment can deliver pressure up to 150-200 MPa.

In conclusion, extensional flow taking place before and in the HP-valve gap probably led to droplet deformation and break-up. Also turbulence, recirculation and cavitation phenomena taking place at the outlet and downstream of the HP valve gap could contribute to additional break up but also to recoalescence of oil droplets.

Brinzolamide solubility in water is pH dependent with minimal solubility at neutral pH and increased solubility at more basic or acidic pH. In order to evaluate Brinzolamide solubilization at different pH DOE were planned. Buffered solution comprises of citric acid monohydrate, hydroxyethyl cellulose (Natrasol 250 HX) and disodium edetate was used as aqueous phase. Different combination of surfactant and co-surfactant (type and concentration) along different manufacturing process were utilized to see the impact of all these combinations on the Brinzolamide solubilization either in micelle or microemulsion with respect to pH.

In micellar solubilization Brinzolamide is dissolved in the mixer of surfactant and cosurfactant and followed by dropwise addition in the aqueous buffer vehicle.

In micro-emulsion Brinzolamide is dissolved in combination of oil, surfactant and co-surfactant followed by dropwise addition in the aqueous buffer vehicle.

The following steps are common for both types of formulations:

Decrease pH Method

In this method aqueous buffer vehicle pH is adjusted to 4.00. Dissolved Brinzolamide phase is added dropwise to the aqueous buffer vehicle by maintain pH below 4.30 by using IN hydrochloric acid. After addition of Brinzolamide phase the bulk is divided equally to six parts and adjusted to different pH.
Part 1—pH adjusted to 5.00
Part 2—pH adjusted to 5.30
Part 3—pH adjusted to 5.50
Part 4—pH adjusted to 6.00
Part 5—pH adjusted to 6.40
Part 6—pH adjusted to 7.00

All the samples were filtered and subjected to HPLC analysis for content of Brinzolamide.

High Pressure Homoginizer (HPH) Method.

In this method aqueous buffer vehicle pH is adjusted to 4.00. Dissolved Brinzolamide phase is added dropwise to the aqueous buffer vehicle. After addition of Brinzolamide phase the bulk is subjected to HPH at a pressure of 1000 bar by maintaining temperature of product below 45° C. After that bulk is divided equally to six parts and adjusted to different pH.
Part 1—pH adjusted to 5.00
Part 2—pH adjusted to 5.30
Part 3—pH adjusted to 5.50
Part 4—pH adjusted to 6.00
Part 5—pH adjusted to 6.40
Part 6—pH adjusted to 7.00

All the samples were filtered and subjected to HPLC analysis for content of Brinzolamide

The following examples illustrate preferred embodiments in accordance with the present invention, without limiting the scope or the spirit of the invention.

### EXAMPLES

### Micro-Emulsion

### Example 1

Primary screening of ophthalmically acceptable excipients was based on their solubilization ability to dissolve drug and their functional role in the final formulation. 10 ml of excipient was taken in glass beaker followed by addition of drug in small increment in order to test the solubilization ability. Primary screening was based on visual observation of the sample. Sample should be free of any visible undissolved particles before addition on another small increment of API. Afterwards, the mixture was heated to reach the saturated solubility of the excipient.

TABLE 1
Solubility study of ophthalmically acceptable excipients

| Commercial Name | Brinzolamide dissolved in 10 g |
|---|---|
| Isopropyl Myristate (Kollicream IPM) | 60 mg |
| PEG200 | 2000 mg |
| Tween 80 | 2000 mg |
| Sesame Oil | 60 mg |
| Silicone Oil | 60 mg |
| Transcutol | 800 mg |
| Anfopon | 60 mg |
| Myritol 318 | 60 mg |
| Dubcare Olga SF | 60 mg |
| Radia 7104 | 60 mg |
| Kollisolv GTA | 210 mg |
| Kolliphor EL | 1015.1 mg |

According to the solubility study results PEG 200, Transcutol, Tween 80 and Kolliphor EL were considered as potential candidates for the composition. The oils used for solubility determination showed very low solubility of drug.

### Example 2

Additional solubility trials were planned in order to see the impact of surfactant/oil combination on solubility of Brinzolamide. 5 g of each of the vehicles of Table 2 was added in 10 ml beakers under stirring, containing excesses of drug. Afterwards, the mixture was heated to improve solubilization. After equilibrium was achieved, the mixture was centrifuged at 4000 rpm for 10 minutes and the supernatant was quantified by high-performance liquid chromatography.

TABLE 2
Solubility study of oil/surfactant combination

| | mg API | mg API | | | |
|---|---|---|---|---|---|
| | dissolved | dissolved | Total mg | API dissolved | Assay |
| Surfactants/Oils | @ RT | temperature | dissolved | mg/ml | (mg/ml) |
| PEG 200 (5 g) | 908.2 | 1042.3 | 1950.5 | 390.1 | 236.63 |
| Kolliphor EL (5 g) | 502.6 | 512.5 | 1015.1 | 203.0 | 177.65 |
| Tween 80 (5 g) | 501.9 | 519.0 | 1020.9 | 204.2 | 169.08 |
| Transcutol (5 g) | 1008.5 | 991.5 | 2.000 | 400.0 | 189.94 |
| Kolliphor EL + IPM (5 g + 1 g) | 802.3 | 200.0 | 1002.3 | 167.1 | 104.70 |
| Kolliphor EL + IPM (5 g + 3 g) | 500.5 | 512.2 | 1012.7 | 126.6 | 76.75 |
| Kolliphor EL + IPM (5 g + 5 g) | 401.3 | 306.7 | 708.0 | 70.8 | 68.97 |
| Tween 80 + IPM (5 g + 1 g) | 400.9 | 401.9 | 802.8 | 133.8 | 113.05 |
| Tween 80 + IPM (5 g + 3 g) | 400.3 | 402.4 | 802.7 | 100.3 | 84.49 |
| Tween 80 + IPM (5 g + 5 g) | 400.0 | 508.6 | 908.6 | 90.9 | 52.08 |
| Tween 80 + IPM + Kolliphor | 501.5 | 507.6 | 1009.1 | 110.9 | 88.93 |
| EL (5 g + 1 g + 3.1 g) | | | | | |
| Tween 80 + IPM + Kolliphor | 404.0 | 602.1 | 1002.1 | 110.1 | 102.12 |
| EL (5 g + 1 g + 4.6 g) | | | | | |
| Tween 80 + IPM + Kolliphor | 403.4 | 402.2 | 805.6 | 43.5 | 39.65 |
| EL (5 g + 1 g + 12.5 g) | | | | | |

Kolliphor EL and Tween 80 showed better solubility alone as well as in combination with IPM.

### Example 3

Based on solubility results Pseudo ternary phase diagrams were prepared to evaluate the microemulsion region. Kolliphor EL and Tween 80 as surfactant, PEG200 and Transcutol as co-surfactant and Isopropyl Myristate (IPM) as oil were used for the Pseudo ternary phase diagrams.

Three phase behavior systems were studied in order to identify the best emulsifying region with respect to amounts of oil, water, surfactant: co-surfactant. The surfactant: co-surfactant combinations studied were Kolliphor EL: PEG 200 and Tween 80: Trancutol both in a ratio 5:2. The oily phase was added to such mixtures at different amounts: 10%, 20%, 30%, 40%, 50%, 60%, 70%, 80% and 90%. Each mixture was then titrated by adding water up to clouding.

Next step was to see the impact of Brinzolamide API in the stability of microemulsion based on Pseudo ternary phase diagrams. API was dissolved in a mixture of oil+surfactant: co-surfactant (Table 3 & 4). Buffer solution of pH 7.20 was added dropwise.

TABLE 3
IPM + Kolliphor EL: PEG200

| | |
|---|---|
| Composition | % w/v |

| | | |
|---|---|---|
| Brinzolamide | 2.50 | 2.30 |
| Isopropyl Myristate (IPM) | 3.25 | 7.50 |
| PEG200 | 8.4 | 8.60 |
| Kolliphor EL | 21.0 | 21.50 |

| | |
|---|---|
| Buffer solution of pH 7.20 | q.s to 100 ml |

TABLE 4
IPM + Tween 80: Transcutol

| | |
|---|---|
| Composition | % w/v |

| | | |
|---|---|---|
| Brinzolamide | 2.50 | 0.50 |
| Isopropyl Myristate (IPM) | 3.20 | 3.30 |
| Tween 80 | 20.00 | 9.50 |
| Transcutol | 8.00 | 3.50 |

| | |
|---|---|
| Buffer solution of pH 7.20 | q.s to 100 ml |

The experiment results showed that the microemulsion obtained was the desirable but the quantity of the surfactant was too high and thus not acceptable.

### Example 4

2 Level Factorial design was performed in order to evaluate the optimum pH for the formulation, the concentration of dissolved Brinzolamide in the solution and the impact of presence of oil in the composition (Table 5)

TABLE 5
Design of experiment

| StdOrder | CenterPt | Blocks | Surfactant | Co-surfactant | Man. Process | % IPM |
|---|---|---|---|---|---|---|
| 14 | 1 | 1 | Tween 80 | Transcutol | HPH | 1 |
| 11 | 1 | 1 | Kolliphor EL | PEG200 | Decrease pH | 1 |
| 1 | 1 | 1 | Kolliphor EL | Transcutol | Decrease pH | 0 |
| 9 | 1 | 1 | KolliphorEL | Transcutol | Decrease pH | 1 |
| 12 | 1 | 1 | Tween 80 | PEG200 | Decrease pH | 1 |
| 6 | 1 | 1 | Tween 80 | Transcutol | HPH | 0 |
| 8 | 1 | 1 | Tween 80 | PEG200 | HPH | 0 |
| 10 | 1 | 1 | Tween 80 | Transcutol | Decrease pH | 1 |
| 7 | 1 | 1 | Kolliphor EL | PEG200 | HPH | 0 |
| 3 | 1 | 1 | Kolliphor EL | PEG200 | Decrease pH | 0 |
| 4 | 1 | 1 | Tween 80 | PEG200 | Decrease pH | 0 |
| 15 | 1 | 1 | Kolliphor EL | PEG200 | HPH | 1 |
| 5 | 1 | 1 | Kolliphor EL | Transcutol | HPH | 0 |
| 13 | 1 | 1 | Kolliphor EL | Transcutol | HPH | 1 |
| 16 | 1 | 1 | Tween 80 | PEG200 | HPH | 1 |
| 2 | 1 | 1 | Tween 80 | Transcutol | Decrease pH | 0 |

Five Factors were Utilized:

1. Type of surfactant: Two different surfactants used (Kolliphor EL and Tween 80) in 7.5% concentration;

2. Type of co-surfactant: Two different co-surfactants used (Transcutol and PEG200) in 2.0% concentration;

3. Manufacturing Process: Two different manufacturing process utilized (Decrease pH and High pressure homogenizer-HPH);

4. % IPM: Two level (0% and 1%) were utilized;

5. Response: Assay at pH 5.50, 5, 5.30, 6, 6.40, 7.

The below conclusions were obtained by measuring the response for all formulations of the experimental design:
Addition of IPM in formulation above pH 6 helps in incorporation of higher amount of drug in formulation;
Oil does not have any significant impact at pH 5.30;
Tween 80 as surfactant, PEG200 as co-surfactant and HPH process are optimum to achieve highest incorporation of Brinzolamide in the formulation.

In Table 6 below are presented the optimum formulations for microemulsion according to the present invention and in Table 7 their stability data. However, only formulations 1 & 2 succeeded in solubilizing 1 g of Brinzolamide and achieved the target of the present invention.

TABLE 6
Optimum formulations 1-6 (Micro-emulsion)

| Compositions | 1 | 2 | 3 | 4 | 5 | 6 |
|---|---|---|---|---|---|---|
| Brinzolamide | 1.000 | 1.000 | 0.50 | 0.50 | 0.38 | 0.33 |
| Kolliphor EL | 5.000 | — | 7.500 | 4.000 | 7.500 | 4.000 |
| Tween 80 | — | 5.000 | — | 3.500 | — | 3.500 |
| Isopropyl Myristate | 1.000 | 1.000 | 1.000 | 1.000 | 1.000 | 1.000 |
| PEG 200 | 2.000 | 2.000 | 2.000 | 2.000 | 2.000 | 2.000 |
| Natrosol 250 HX | 0.250 | 0.250 | 0.250 | 0.250 | 0.250 | 0.250 |
| Disodium Edetate | 0.010 | 0.010 | 0.010 | 0.010 | 0.010 | 0.010 |
| Sodium Citrate Dihydrate | 0.294 | 0.294 | 0.294 | 0.294 | 0.294 | 0.294 |
| Benzalkonium Chloride | 0.020 | 0.020 | 0.020 | 0.020 | 0.020 | 0.020 |
| 50% w/w | | | | | | |
| NaOH and/or HCl | q.s. to adjust pH | | | | | |
| water for injection | q.s. to 100 ml | | | | | |
| Final pH | 5.00 | 5.00 | 6.00 | 6.00 | 6.40 | 7.00 |

TABLE 7
Stability data

| | | | | | | |
|---|---|---|---|---|---|---|
| Time Interval | COMP 1 | COMP 2 | COMP 3 | COMP 4 | COMP 5 | COMP 6 |

Assay of Brinzolamide

| | | | | | | |
|---|---|---|---|---|---|---|
| Zero Time | 103.6 | 104.6 | 103.0 | 104.7 | 103.3 | 104.5 |
| 25° C. 6 Months | 103.1 | 104.2 | 103.7 | 104.4 | 103.0 | 104.2 |
| 40° C. 6 Months | 103.3 | 104.5 | 103.1 | 103.9 | 103.8 | 104.6 |

Total Impurity

| | | | | | | |
|---|---|---|---|---|---|---|
| Zero Time | 0.28 | 0.21 | 0.26 | 0.27 | 0.24 | 0.25 |
| 25° C. 6 Months | 0.28 | 0.33 | 0.33 | 0.34 | 0.32 | 0.31 |
| 40° C. 6 Months | 0.74 | 0.79 | 0.79 | 0.77 | 0.79 | 0.76 |

IMP A (RRT 0.83)

| | | | | | | |
|---|---|---|---|---|---|---|
| Zero Time | 0.01 | ND | ND | ND | ND | ND |
| 25° C. 6 Months | ND | ND | 0.01 | ND | ND | ND |
| 40° C. 6 Months | 0.02 | 0.02 | 0.02 | 0.02 | 0.02 | 0.02 |

IMP B (RRT 0.65)

| | | | | | | |
|---|---|---|---|---|---|---|
| Zero Time | 0.07 | 0.07 | 0.07 | 0.07 | 0.06 | 0.06 |
| 25° C. 6 Months | 0.10 | 0.08 | 0.10 | 0.09 | 0.08 | 0.10 |
| 40° C. 6 Months | 0.15 | 0.15 | 0.16 | 0.16 | 0.15 | 0.16 |

### Micellar Solubilization

### Example 5

2 Level Factorial design was performed in order to evaluate the optimum pH and impact of the formulation variables on the micellar solubilization of Brinzolamide. (Table 8)

TABLE 8
Design of experiment

| | | | Man. | % | % |
|---|---|---|---|---|---|
| StdOrder | Surfactant | Co-surfactant | Process | Surfactant | Co-surfactant |
| 16 | Tween 80 | PEG200 | HPH | 10 | 2.5 |
| 6 | Tween 80 | Transcutol | HPH | 5 | 2.5 |
| 24 | Tween 80 | PEG200 | HPH | 7.5 | 2 |
| 2 | Tween 80 | Transcutol | Decrease pH | 5 | 1.5 |
| 17 | Kolliphor EL | Transcutol | Decrease pH | 7.5 | 2 |
| 12 | Tween 80 | PEG200 | Decrease pH | 10 | 1.5 |
| 9 | Kolliphor EL | Transcutol | Decrease pH | 10 | 1.5 |
| 5 | Kolliphor EL | Transcutol | HPH | 5 | 1.5 |
| 22 | Tween 80 | Transcutol | HPH | 7.5 | 2 |
| 7 | Kolliphor EL | PEG200 | HPH | 5 | 2.5 |
| 18 | Tween 80 | Transcutol | Decrease pH | 7.5 | 2 |
| 13 | Kolliphor EL | Transcutol | HPH | 10 | 2.5 |
| 4 | Tween 80 | PEG200 | Decrease pH | 5 | 2.5 |
| 23 | Kolliphor EL | PEG200 | HPH | 7.5 | 2 |
| 1 | Kolliphor EL | Transcutol | Decrease pH | 5 | 2.5 |
| 14 | Tween 80 | Transcutol | HPH | 10 | 1.5 |
| 10 | Tween 80 | Transcutol | Decrease pH | 10 | 2.5 |
| 20 | Tween 80 | PEG200 | Decrease pH | 7.5 | 2 |
| 8 | Tween 80 | PEG200 | HPH | 5 | 1.5 |
| 3 | Kolliphor EL | PEG200 | Decrease pH | 5 | 1.5 |
| 21 | Kolliphor EL | Transcutol | HPH | 7.5 | 2 |
| 19 | Kolliphor EL | PEG200 | Decrease pH | 7.5 | 2 |
| 15 | Kolliphor EL | PEG200 | HPH | 10 | 1.5 |
| 11 | KolliphorEL | PEG200 | Decrease pH | 10 | 2.5 |

Six factors were utilized:

1. Type of surfactant: Two different surfactants used (Kolliphor EL and Tween 80);

2. Type of co-surfactant: Two different co-surfactants used (Transcutol and PEG200);

3. Manufacturing Process: Two different manufacturing process utilized (Decrease pH and High pressure homogenizer-HPH);

4. % of surfactant: Two level (5% and 10%) were utilized with one center point for the design;

5. % of co-surfactant: Two level (1.5% and 2.5%) were utilized with one center point for the design;

6. Response: Assay at pH 5, 5.3, 5.5, 6, 6.4, 7.

The below conclusions were obtained by measuring the response for all formulations of the experimental design:
Between pH 6 to 7 micellar solubilization of Brinzolamide is totally dependent on the concentration of the surfactant;
At pH 5 micellar solubilization depends on type of surfactant, % of surfactant and % of co-surfactant;
At pH 5.30 micellar solubilization depends on % of surfactant and type of co-surfactant;
At pH 5.50 micellar solubilization depends on % of surfactant and type of co-surfactant and manufacturing process utilized;
For micellar solubilization of 1.00% w/v brinzolamide target pH is 5.00 to 5.50. PEG200 was utilized as co-surfactant as it showed better results as at pH 5.30 and 5.50.

In Table 9 below are presented the optimum formulations for micellar solubilization according to the present invention and in Table 10 their stability data. However, only formulations 1-6 succeeded in solubilizing 1 g of Brinzolamide and achieved the target of the present invention.

TABLE 9
Optimum formulations 1-9 (Micellar solubilization)

| Ingredients | Comp 1 | Comp 2 | Comp 3 | Comp 4 | Comp 5 | Comp 6 | Comp 7 | Comp 8 | Comp [symbol] |
|---|---|---|---|---|---|---|---|---|---|
| Brinzolamide | 1.000 | 1.000 | 1.000 | 1.000 | 1.000 | 1.000 | 0.38 | 0.32 | 0.27 |
| Kolliphor EL | 5.000 | 7.500 | 5.000 | — | — | 3.750 | 7.500 | 7.500 | 7.500 |
| Tween 80 | — | — | 5.000 | 5.000 | 7.500 | 3.750 | — | — | — |
| PEG 200 | 2.000 | 1.500 | 1.000 | 2.000 | 1.500 | 1.000 | 2.000 | 2.000 | 2.000 |
| [symbol] atrosol 250 HX | 0.250 | 0.250 | 0.250 | 0.250 | 0.250 | 0.250 | 0.250 | 0.250 | 0.250 |
| [symbol] isodium Edetate | 0.010 | 0.010 | 0.010 | 0.010 | 0.010 | 0.010 | 0.010 | 0.010 | 0.010 |
| Sodium Citrate | 0.294 | 0.294 | 0.294 | 0.294 | 0.294 | 0.294 | 0.294 | 0.294 | 0.294 |
| Dihydrate | | | | | | | | | |
| Benzalkonium | 0.020 | 0.020 | 0.020 | 0.020 | 0.020 | 0.020 | 0.020 | 0.020 | 0.020 |
| Chloride | | | | | | | | | |
| 50% w/w | | | | | | | | | |

| | | | | |
|---|---|---|---|---|
| Sodium | q.s. to adjust pH to 5.00 | q.s. to | q.s. to | q.s. to |
| [symbol] ydroxide and/or | | adjust | adjust | adjust |
| [symbol] ydrochloric acid | | pH to | pH to | pH to |
| | | 6.00 | 6.40 | 7.00 |

| | |
|---|---|
| water for | q.s. to 100 ml |
| injection | |

[symbol]

indicates data missing or illegible when filed

TABLE 10
Stability data

| | | | | | | | | | |
|---|---|---|---|---|---|---|---|---|---|
| Time Interval | COMP 1 | COMP 2 | COMP 3 | COMP 4 | COMP 5 | COMP 6 | COMP 7 | COMP 8 | COMP 9 |

Assay of Brinzolamide

| | | | | | | | | | |
|---|---|---|---|---|---|---|---|---|---|
| Zero Time | 98.4 | 98.3 | 98.3 | 97.8 | 98.6 | 98.2 | 98.1 | 98.2 | 97.9 |
| 25° C. 6 Months | 99.9 | 98.5 | 98.4 | 98.7 | 99.4 | 98.4 | 98.3 | 98.2 | 98.5 |
| 40° C. 6 Months | 97.7 | 98.3 | 98.7 | 97.7 | 99.0 | 98.3 | 98.2 | 98.6 | 97.8 |

Total Impurity

| | | | | | | | | | |
|---|---|---|---|---|---|---|---|---|---|
| Zero Time | 0.80 | 0.78 | 0.71 | 0.71 | 0.76 | 0.70 | 0.78 | 0.87 | 0.91 |
| 25° C. 6 | 1.80 | 1.82 | 1.74 | 1.62 | 1.71 | 1.81 | 1.76 | 1.69 | 1.59 |
| Months | | | | | | | | | |
| 40° C. 6 | 2.19 | 2.42 | 2.46 | 2.76 | 2.57 | 2.76 | 2.37 | 2.40 | 2.67 |
| Months | | | | | | | | | |

IMP A (RRT 0.83)

| | | | | | | | | | |
|---|---|---|---|---|---|---|---|---|---|
| Zero Time | 0.02 | 0.02 | 0.02 | 0.01 | 0.02 | 0.02 | 0.02 | 0.02 | 0.01 |
| 25° C. 6 | 0.02 | 0.04 | 0.02 | 0.01 | 0.02 | 0.04 | 0.04 | 0.02 | 0.01 |
| Months | | | | | | | | | |
| 40° C. 6 | 0.14 | 0.13 | 0.11 | 0.18 | 0.10 | 0.18 | 0.12 | 0.10 | 0.16 |
| Months | | | | | | | | | |

IMP B (RRT 0.65)

| | | | | | | | | | |
|---|---|---|---|---|---|---|---|---|---|
| Zero Time | 0.36 | 0.33 | 0.30 | 0.39 | 0.33 | 0.30 | 0.32 | 0.31 | 0.38 |
| 25° C. 6 | 0.47 | 0.43 | 0.48 | 0.51 | 0.44 | 0.47 | 0.42 | 0.47 | 0.50 |
| Months | | | | | | | | | |
| 40° C. 6 | 0.66 | 0.61 | 0.67 | 0.64 | 0.59 | 0.70 | 0.60 | 0.65 | 0.63 |
| Months | | | | | | | | | |

While the invention has been described with reference to various specific and preferred embodiments and examples, it should be however understood that variations and modifications may be made without departing from the spirit and scope of the invention as defined in the appended claims.

1. A process for the preparation of a stable ophthalmic composition of Brinzolamide comprising the step of forming an active pharmaceutically ingredient phase by dissolving Brinzolamide in a mixture comprising surfactant, co-surfactant and optionally oil.

2. The process according to claim 1, comprising the step of adding slowly the Brinzolamide phase to an aqueous buffer vehicle having pH maintained below 4.30.

3. The process according to claim 2 optionally comprising the step of subjecting the bulk to a pressure of 1000 bar by maintaining temperature of product below 45° C.

4. The process according to claim 2, wherein pH is adjusted to 5-7.

5. The process according to claim 1, wherein the oil is selected from isopropyl myristate, medium chain triglycerides, vegetable oils.

6. The process according to claim 1, wherein the surfactant is selected from polyethoxylated castor oil, polysorbates, quaternary ammonium compounds, fatty acids.

7. The process according to claim 1, wherein the co-surfactant is selected from 2-(2-ethoxyethoxy)-ethanol, polyethylene glycols.

8. The process according to claim 1, further comprising a chelating agent selected from disodium edetate, citric acid and salts thereof.

9. The process according to claim 1, further comprising a water soluble polymer selected from hydroxyethyl cellulose, polyvinyl alcohol, povidone.

10. The process according to claim 2, wherein the aqueous buffer vehicle comprises citric acid monohydrate, hydroxyethyl cellulose and disodium edetate.

11. The process according to claim 1, further comprising a pH-adjusting agent selected from acetic acid, hydrochloric acid, sodium hydroxide, citric acid or salts thereof.

12. The process according to claim 1, comprising a preservative selected from benzalkonium chloride, borate, polyquateraium-1.
